(19) United States
(12) Patent Application Publication
(10) Pub. No.: US 2007/0134303 A1
(43) Pub. Date: Jun. 14, 2007

(54) Protective and therapeutic article

(76) Inventors: Ali Yahiaoui, Roswell, GA (US); Kaiyuan Yang, Cumming, GA (US)

Correspondence Address: KIMBERLY-CLARK WORLDWIDE, INC., 401 NORTH LAKE STREET, NEENAH, WI, 54956, US

(21) Appl. No.: 11/302,644

(22) Filed: Dec. 14, 2005

(51) Int. Cl. A61K 38/44; A61K 38/05; A61K 9/70

(52) U.S. Cl. 424/443; 424/94.4; 514/18; 442/123

(57) ABSTRACT

An antimicrobial protective article is described. The article has a first, outer surface that is coated at least partially with an antimicrobial agent containing polyhexamethylene biguanide (PHMB), and at least one or a combination of the following: chitosan, a chaotropic surfactant, polyols, copper oxide, and an organic acid, and a second, inner surface adapted to contact a wearer's skin that has a therapeutic agent. The present protective article can adapted for used in a variety of applications in the home or resort spas.

### FIELD OF INVENTION

The present invention relates to a protective article that is adapted to be worn on a person's body. The article has a substrate body composed largely of an elastic, flexible nonwoven material with an intermediate layer. The article provides antimicrobial and/or antiviral protection as well as therapeutic skin health benefits.

### BACKGROUND OF THE INVENTION

In recent years, the consumer has become more conscious of antimicrobial precautions and the need to follow good hygiene and avoid the transmission of hazardous microbes. Various kinds of products have been developed to address this need from soaps to protective body wear. Over the years, manufacturers have developed many types and styles of articles, such as gloves, gowns, or other garments to protect a wear's body. Depending on the type of environment, nature of work, or desired properties, these articles can be made from a variety of materials, including woven cloth fabrics, leather, natural latex or synthetic polymer elastomeric materials, or combinations of such materials.

A need exists however for a consumer article that can protect the user from bacteria or viral contamination, as well as be therapeutic to the area of the user's body that the article contacts. Such a product would be welcome in either the home or spas.

### SUMMARY OF THE INVENTION

The present invention pertains to a protective article that can be used in a variety of settings. The article has a body formed in major part with a nonwoven fiber web having cross-direction and machine direction stretchability. The body is coated with an antimicrobial or antiviral composition over at least a portion of a first, exterior surface of the nonwoven fiber web. The antimicrobial or antiviral composition is non-cytotoxic active but exhibits a broad spectrum kill efficacy. A therapeutic composition is applied to a second, interior surface adapted to contact human or mammalian skin. The article can have a variety of configurations, including, but not limited to external garments, gloves, socks, booties or other foot coverings, elbow or knee wraps or padding, face masks, ear and head coverings.

Additional features and advantages of the present protective and/or sanitizing articles and associated methods of manufacture will be disclosed in the following detailed description. It is understood that both the foregoing summary and the following detailed description and examples are merely representative of the invention, and are intended to provide an overview for understanding the invention as claimed.

### BRIEF DESCRIPTION OF THE DRAWINGS

FIGS. 1A and 1B are perspective views of a glove, according to an embodiment of the present invention, the shaded areas on an outer surface of the glove represent areas treated with an antimicrobial composition;

FIG. 2 is an exploded perspective view of one embodiment of a stretch-bonded laminate that may be used in accordance with the present disclosure;

FIG. 3 is an exploded perspective view of another embodiment of a stretch-bonded laminate that may be used in accordance with the present disclosure;

FIG. 4 is an exploded perspective view of still another embodiment of a stretch-bonded laminate that may be used in accordance with the present disclosure;

FIG. 5 is a perspective view of the stretch-bonded laminate illustrated in FIG. 2;

Repeat use of reference characters in the present specification and drawings is intended to represent the same or analogous features or elements of the invention.

### DETAILED DESCRIPTION

### Section I—Definitions

In this specification and the appended claims, the singular forms “a,” “an,” and “the” include plural reference unless the context clearly dictates otherwise. Unless defined otherwise, all technical and scientific terms used herein have the same meaning as commonly understood or generally accepted by one of ordinary skill in the art to which this invention pertains.

The term “coform” as used herein refers to a meltblown material to which at least one other material is added during the meltblown material formation. The meltblown material may be made of various polymers, including elastomeric polymers. Various additional materials may be added to the meltblown fibers during formation, including, for example, pulp, superabsorbent particles, cellulose or staple fibers. Coform processes are illustrated in commonly assigned U.S. Pat. No. 4,818,464 to Lau, and U.S. Pat. No. 4,100,324 to Anderson et al., the entire contents of which are incorporated herein by reference in their entirety for all purposes.

The term “elastic” refers to any material, including a film, fiber, nonwoven web, or combination thereof, which upon application of a biasing force, is stretchable to a stretched, biased length which is at least about 150 percent, or one and a half times, its relaxed, unstretched length, and which will recover at least 15 percent of its elongation upon release of the stretching, biasing force.

The terms “elastomer” or “elastomeric” refer to polymeric materials that have properties of stretchability and recovery.

The term “meltblown fibers” refers to fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al., the entire contents of which are incorporated herein by reference in their entirety for all purposes. Meltblown fibers are microfibers that may be continuous or discontinuous with diameters generally less than 10 microns.

The terms “necking” or “neck stretching” interchangeably refer to a method of elongating a nonwoven fabric, generally in the machine direction, to reduce its width (cross-machine direction) in a controlled manner to a desired amount. The controlled stretching may take place under cool, room temperature or greater temperatures and is limited to an increase in overall dimension in the direction being stretched up to the elongation required to break the fabric, which in most cases is about 1.2 to 1.6 times. When relaxed, the web retracts toward, but does not return to, its original dimensions. Such a process is disclosed, for example, in U.S. Pat. No. 4,443,513 to Meitner and Notheis, U.S. Pat. Nos. 4,965,122, 4,981,747 and 5,114,781 to Morman, and U.S. Pat. No. 5,244,482 to Hassenboehler Jr. et al., the entire contents of which are incorporated herein by reference in their entirety for all purposes.

The term “neck bonded laminate” refers to a composite material having at least two layers in which one layer is a necked, non-elastic layer and the other layer is an elastic layer. The layers are joined together when the non-elastic layer is in an extended (necked) condition. Examples of neck-bonded laminates are such as those described in U.S. Pat. Nos. 5,226,992, 4,981,747, 4,965,122 and 5,336,545 to Morman, the entire contents of which are incorporated herein by reference in their entirety for all purposes.

The term “nonwoven fabric or web” as used herein refers to a web having a structure of individual fibers or threads which are interlaid, but not in an identifiable manner as in a knitted fabric. Nonwoven fabrics or webs have been formed from various processes such as, for example, meltblowing processes, spunbonding processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91).

The term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the molecule. These configurations include, but are not limited to isotactic, syndiotactic and random symmetries.

The term “recover” or “recovery” refers to a contraction of a stretched material upon termination of a biasing force following stretching of the material by application of the biasing force. For example, if a material having a relaxed, unbiased length of one (1) inch is elongated 50 percent by stretching to a length of one and one half (1.5) inches the material would have a stretched length that is 150 percent of its relaxed length. If this exemplary stretched material contracted, that is recovered to a length of one and one tenth (1.1) inches after release of the biasing and stretching force, the material would have recovered 80 percent (0.4 inch) of its elongation.

The term “reversibly necked material” refers to a material that possesses stretch and recovery characteristics formed by necking a material, then heating the necked material, and cooling the material. Such a process is disclosed in U.S. Pat. No. 4,965,122 to Morman, commonly assigned to the assignee of the present invention, the entire contents of which are incorporated by reference herein in its entirety for all purposes.

The term “spunbonded fibers” refers to small diameter fibers that are formed by extruding molten thermoplastic material as filaments from a plurality of fine, usually circular capillaries of a spinneret with the diameter of the extruded filaments then being rapidly reduced to fibers as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al., the entire contents of which are incorporated herein by reference in their entirety for all purposes. Spunbond fibers can be continuous and have diameters generally greater than about 7 microns, more particularly, between about 10 and about 20 microns.

The term “stretch” refers to the ability of a material to extend upon application of a biasing force. Percent stretch is the difference between the initial dimension of a material and that same dimension after the material has been stretched or extended following the application of a biasing force. Percent stretch may be expressed as [(stretched length+initial sample length)/initial sample length]×100. For example, if a material having an initial length of one (1) inch is stretched 0.50 inch, that is, to an extended length of 1.50 inches, the material can be said to have a stretch of 50 percent.

The term “stretch-bonded laminate” refers to a composite material having at least two layers in which one layer is a gatherable layer and the other layer is an elastic layer. The layers are joined together when the elastic layer is extended from its original condition so that upon relaxing the layers, the gatherable layer is gathered. Such a multilayer composite elastic material may be stretched to the extent that the material gathered between the bond locations allows the elastic material to elongate. One type of stretch-bonded laminate is disclosed, for example, by U.S. Pat. No. 4,720,415 to Vander Wielen et al., the entire contents of which are incorporated herein by reference in its entirety for all purposes. Other composite elastic materials are disclosed in U.S. Pat. No. 4,789,699 to Kieffer et al., U.S. Pat. No. 4,781,966 to Taylor and U.S. Pat. Nos. 4,657,802 and 4,652,487 to Morman and U.S. Pat. No. 4,655,760 to Morman et al., the contents of which are incorporated herein by reference in their entirety.

The term “ultrasonic bonding” refers to a process in which materials (fibers, webs, films, etc.) are joined by passing the materials between a sonic horn and anvil surface, such as a roll. An example of such a process is illustrated in U.S. Pat. No. 4,374,888 to Bornslaeger, the entire contents of which are incorporated herein by reference in their entirety for all purposes.

### Section II—Description

In general, the present invention is directed to flexible, relatively soft nonwoven protective article. The protective article has a nonwoven fiber web having cross-direction and machine direction stretchability with a first side and outer surface, and a second side and inner surface. The nonwoven fiber web can also include a barrier film layer. The first side can be treated with a coating of a stable, non-leaching antimicrobial or antiviral composition over at least a portion of the first, exterior surface of the nonwoven fiber web. The antimicrobial or antiviral composition has a non-cytotoxic activity but a broad spectrum kill. Additionally, a therapeutic composition or treatment can be applied to at least part of the second, interior surface, which is adapted to contact human or other mammalian skin. The therapeutic composition may contain skin-care products, emollients, or other therapeutic agents or compounds that can be delivered by transdermal deliver or application. The article is elastic and stretchy so as to fit snuggly against the wearer's body, but without undue binding or discomfort.

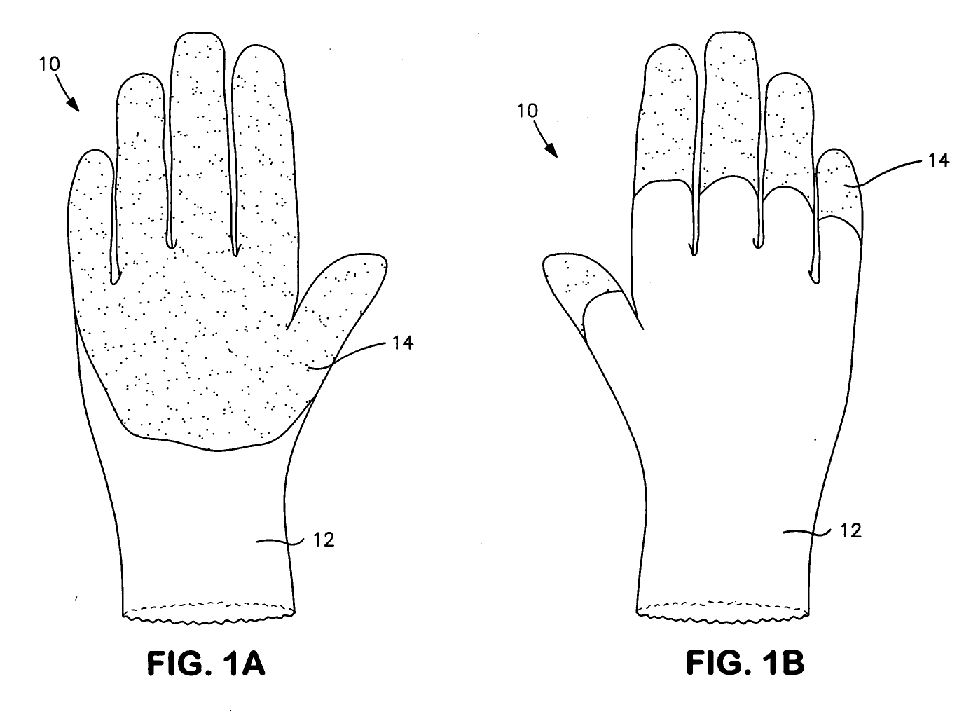

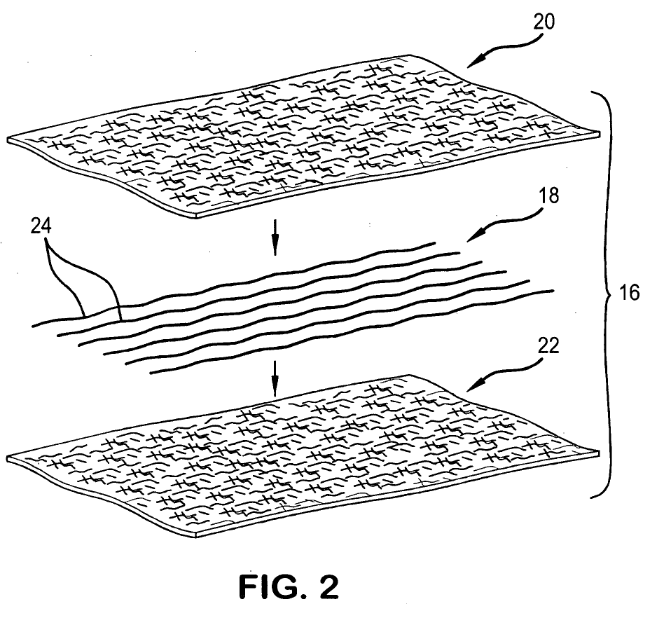

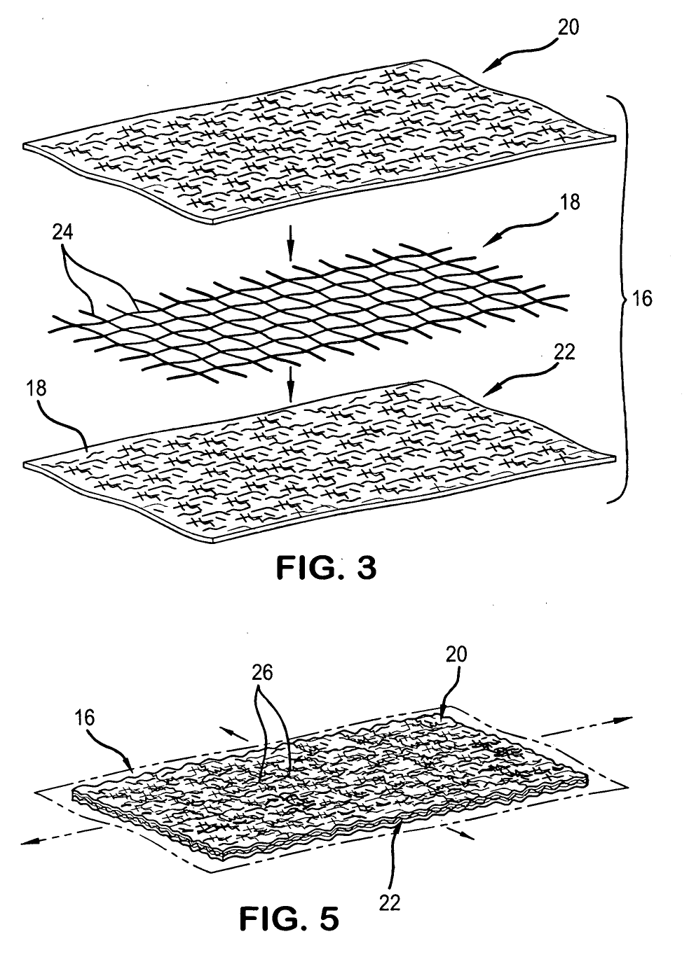

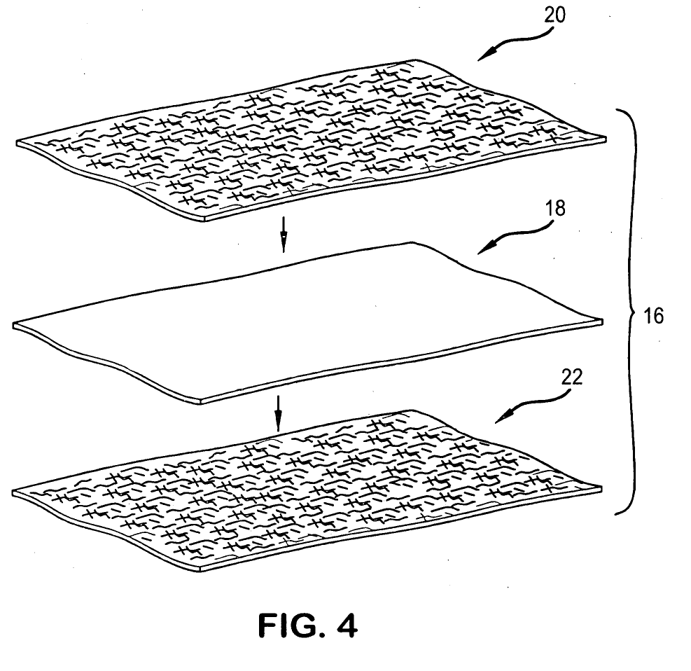

Although the following description refers to gloves, for purpose of illustration, the particular features of the invention can be applied generally to similar articles and protective garments can be constructed or made predominately with nonwoven materials and having a antimicrobial or antiviral coating. In other words, although the specific dimensions or configuration of the garment may change, mutatis mutandis, the common features will remain. FIG. 1 is a representation of a glove 10 according to an iteration of the invention. The glove 10 has an exterior surface 12 that is at least partially treated with an antimicrobial or antiviral agent 14. The glove can be formed from a nonwoven fiber web 16. The nonwoven web 16 is made from a laminate of a first nonwoven layer 20, an intermediate layer 18, and a second nonwoven layer 22, such as illustrated in FIGS. 2-5. The first and second nonwoven layers each can be made from either different materials or they can be similar or identical materials. The outside facing 26 of the nonwoven web can be textured to provide good friction and grip, in the case of gloves or foot coverings, while the inner or user contacting surface can be softer, smoother, and more cloth-like. The intermediate layer 18, can be a structural support having uni-directional or bidirectional ribbing, such as show in FIGS. 2 and 3, respectively, or it can be a barrier film layer, such as in FIG. 4. The barrier layer in the nonwoven laminate should permit separation of the inner and outer surfaces and enable one to coat each side of the substrate with different substances easier. A barrier layer is preferred but not required, as one can coat both sides with different formulations without a barrier layer. The articles are relatively inexpensive to produce and have good durability and elastic properties, which can make them appealing for spa-care, therapeutic, as well as other uses.

The elastic laminate may comprise, for instance, a spunbond laminate, a neck-bonded laminate, and mixtures thereof. In one embodiment, for instance, the elastic laminate may have at least three layers. The three layers may include two outer nonwoven layers and a middle layer comprising elastic filaments, an elastic film, or an elastic nonwoven. If desired, the outer layers may be attached to the middle layer while the middle layer is in a stretched state such that the outer layers gather when the middle layer is in a relaxed state. The outer layers may comprise the same or different materials. For example, the outer layers may comprise spunbond webs, meltblown webs, coform webs and laminates thereof. In one embodiment, the outer layer forming the exterior surface of the hollow member may comprise a meltblown web, while the outer layer of the elastic laminate forming the interior surface of the hollow member may comprise a spunbond web. The elastic laminate may have any suitable basis weight depending upon desired characteristics of the garment and its intended uses. The basis weight of the elastic laminate may vary, for instance, from about 20 gsm to about 400 gsm or greater.

Alternatively, the article may include a first panel attached to a second panel along a seam. The seam, for instance, may have a thickness of less than about 1 mm to about 4 mm and may be formed by ultrasonically bonding the first panel to the second panel. Each panel may comprise a similar elastic laminate or different elastic laminates. For example, in one embodiment, one panel may comprise a neck-bonded laminate, while the second panel may comprise a spunbond laminate. For instance, in this embodiment, the neck-bonded laminate having one dimensional stretch characteristics may comprise a palm portion of the glove while a stretch-bonded laminate having two dimensional stretch characteristics may form a back portion of the glove. Alternatively, one can employ a single panel that is folded or rolled in upon itself and secured along a common edge, either end to end abutting or slightly overlapping.

A protective article can take the form of a variety of configurations, including, but not limited to external garments, gloves, socks or other foot coverings, elbow or knee wraps or padding, face masks, ear and head coverings. A garment can have a shape to fit over an extremity such as a hand, arm, foot, or leg, for instance, a glove, sleeve, or sock. In other iterations, the garment can be a jacket, pant, robe, or gown, which covers or surrounds at least a portion of a wearer's body. Glove iterations can be employed in clinical or hospital environments, or in every day activities for light dusting, cleaning, shopping, driving, playing, and the like.

For instance, a glove, foot wear, or sleeve may comprise a hollow member or body defining an opening for receiving a particular body part therein. In the case of a glove, the hollow member has an interior surface configured to be placed adjacent to a hand when the glove is donned and an opposite exterior surface. The hollow member can have an elastic laminate including at least one nonwoven layer. An antimicrobial/antiviral coating covers at least a portion of the exterior surface of the hollow member. Alternatively, one may wish to have an antimicrobial-antiviral coating present on the interior surface of the hollow member. In some embodiments, a skin cleaning or general cleaning agent can be applied either to at least a portion of either the exterior or interior surfaces of the hollow member. The inner surface has a soft, cloth-like feel against the wear's skin. In some embodiments, the inner surface is tufted to convey a cotton-fiber-like feel.

Synthetic fibers or filaments used in making the nonwoven materials of the base web have any suitable morphology that may include hollow or solid, straight or crimped, single component, conjugate or biconstituent fibers or filaments, and blends or mixtures of such fibers and/or filaments, as are well known in the art. The synthetic fibers used in the present invention may be formed from a variety of thermoplastic polymers where the term “thermoplastic polymer” refers to a long chain polymer that repeatedly softens when exposed to heat and substantially returns to its original state when cooled to ambient temperature. As used herein, the term “polymer” generally includes, but is not limited to, homopolymers, copolymers, such as for example, block, graft, random, and alternating copolymers, terpolymers, etc., and blends and modifications thereof. As used herein, the term “blend” means a mixture of two or more polymers. Furthermore, unless otherwise specifically limited, the term “polymer” shall include all possible geometrical configurations of the molecule. These configurations include, but are not limited to, isotatic, synditatic, and random symmetries.

Exemplary thermoplastics include, without limitation, poly(vinyl) chlorides, polyesters, polyamides, polyfluorocarbons, polyolefins, polyurethanes, polystyrenes, poly(vinyl) alcohols, caprolactams, and copolymers of the foregoing, and elastomeric polymers such as elastic polyolefins, copolyether esters, polyamide polyether block copolymers, ethylene vinyl acetates (EVA), block copolymers having the general formula A-B-A′ or A-B like copoly(styrene/ethylene-butylene), styrene-poly(ethylene-propylene)-styrene, styrene-poly(ethylene-butylene)-styrene, (polystyrene/poly(ethylene-butylene)/polystyrene, poly(styrene/ethylene-butylene/styrene), A-B-A-B tetrablock copolymers and the like.

Many polyolefins are available for fiber production, for example polyethylenes such as Dow Chemical's PE XU 61800.41 linear low density polyethylene (“LLDPE”) and 25355 and 12350 high density polyethylene (“HDPE”) are such suitable polymers. Fiber-forming polypropylenes include Exxon Chemical Company's Escorene7 PD 3445 polypropylene and Montell Chemical Co.'s PF-304 and PF-015. Many other conventional polyolefins are commercially available and include polybutylenes and others.

Examples of polyamides and their methods of synthesis may be found in “Polymer Resins” by Don E. Floyd (Library of Congress Catalog No. 66-20811, Reinhold Publishing, New York, 1966). Particularly commercially useful polyamides are nylon-6, nylon 6,6, nylon-11 and nylon-12. These polyamides are available from a number of sources such as Emser Industries of Sumter, South Carolina (Grilon7 & Grilamid7 nylons), Atochem Inc. Polymers Division of Glen Rock, New Jersey (Rilsan7 nylons), Nyltech of Manchester, N.H. (grade 2169, Nylon 6), and Custom Resins of Henderson, Kentucky (Nylene 401-D), among others.

As stated above, synthetic fibers added to the base web can also include staple fibers which are added to increase the strength, bulk, softness and smoothness of the base sheet. Staple fibers can include, for instance, various polyolefin fibers, polyester fibers, nylon fibers, polyvinyl acetate fibers, cotton fibers, rayon fibers, non-woody plant fibers, and mixtures thereof. In general, staple fibers are typically longer than pulp fibers. For instance, staple fibers typically have fiber lengths of 5 mm and greater. Staple fibers can increase the strength and softness of the final product.

The antimicrobial or antiviral coating may include a formulation having polyhexamethylene biguanide (PHMB), and at least one or a combination of the following active agents: chitosan, a chaotropic surfactant, polyols, copper oxide, and an organic acid. The chaotropic surfactant includes an alkyl-polyglycoside. The polyol includes xylitol. The organic acid can be either: benzoic acid, citric acid, acetic acid, ascorbic acid, glycolic acid, lauric acid, maleic acid, succinic acid, salicylic acid, or a combination thereof. The copper oxide can be either cupric or cuprous oxide, or both. The antimicrobial coating is both chemically and mechanically stable; hence, it does not leach from the substrate surface upon which the coating is applied. This feature reduces the degradation of the amount of antimicrobial or antiviral agents on the substrate surface, which unlike other products can loose significantly their antimicrobial potency over time. Another benefit of stable non-leaching antimicrobial coating is to reduce the probability of resistant strains of bacteria arising over time. Further description, detailed examples, and other iterations of the antimicrobial/antiviral formulations used herein can be found in U.S. patent applications Ser. Nos. 11/216,341 or 11/216,800, the pertinent content of which is incorporated herein by reference.

To provide therapeutic benefits to the wear's skin, a variety of skin care formulations or chemicals can be applied to the protective articles of the present invention. In one embodiment, such as for gloves or face masks, one can apply on the interior of the article aromatherapy agents, such as described in U.S. Patent Publication 2005/0125923, the pertinent contents of which are incorporated herein by refer. When used to address wounds, cuts, bruises, blisters, dry skin, etc., for example, the article can take the form of a patch, glove, appendage sleeve, foot cover, or mask. The present invention can generally include any additive commonly used as healing or pain-killing agents, particularly those which are currently used on conventional appendage bandages. Examples of such additives can include, but are not limited to, anti-inflammatory agents, moisturizing agents, cationic polymers, vasodilators, corticosteroids, dimethyl sulfoxide (DMSO), capsaicin, menthol, methyl salicylate, DMSO/capsaicin, cationic polymers, anti-fungal agents, and the like. For instance, the product can generally include topical analgesics (e.g. BEN-GAY). Additionally, antioxidant agents for anti-aging or wrinkle reduction uses, such as super-oxide dismutase (SOD) or glutathione may also be applied to the inner surface and contacted to skin. These natural human enzymes are produced in abundance in young skin. With age, however, one loses the ability to produce these antioxidant enzymes. Therefore, external replenishment is desired. Alternatively, inorganic antioxidants can also be used, such as selenium and its derivatives in small therapeutic amounts such as in food supplements.

Cationic polymers can help clean wounds because they typically have a strong attraction for negatively charged bacteria and deleterious acidic byproducts. One example of a cationic polymer that is suitable for use in the present invention is chitosan (poly-N-acetylglucosamine, a derivative of chitin) or chitosan salts. Chitosan and its salts are natural biopolymers that can have both hemostatic and bacteriostatic properties. As a result, chitosan can help reduce bleeding and infection. In addition to chitosan and chitosan salts, any other cationic polymers, such as cationic starches (e.g. COBOND made by National Starch) or oligomeric compounds can be used. In some embodiments, combinations of cationic materials can be utilized. In addition, when used as a sleeve for treating other ailments, such as arthritis; “black toe”, “trigger finger”; or jammed, sprained, hyper-extended, dislocated, or broken appendages, an appendage sleeve of the present invention can generally include any additive commonly used to treat such ailments. Examples of such additives can include, but are not limited to, topical analgesics (e.g. BEN-GAY), anti-inflammatory agents, vasodilators, corticosteroids, dimethyl sulfoxide (DMSO), capsaicin, menthol, methyl salicylate, DMSO/capsaicin, cationic polymers, anti-fungal agents, and the like. For instance, suitable anti-inflammatory agents can include any cyclooxygenase-1 (COX-1) or cyclooxygenase-2 (COX-2) inhibitors.

In general, the chemical additives described above can be applied to an appendage sleeve of the present invention according to a number of ways known in the art. For example, the additives can be applied to the sleeve using a saturant system, such as disclosed in U.S. Pat. No. 5,486,381 to Cleveland et al., which is incorporated herein by reference. Moreover, the additives can also be applied by various other methods, such as print, blade, roll, spray, spray-drying, foam, brush treating applications, etc., which are well known in the art. The additives can further be applied as a mixture of molten solids or co-extruded onto the sleeve. Additionally, in another embodiment, the chemical additives can be impregnated into the material during manufacturing as is well known in the art. It should be understood that when coated onto a sleeve as described above, the additives can be applied to the base web before or after the base web is stamped or bonded to form an appendage sleeve of the present invention. Furthermore, if desired, it should also be understood that various additives, solutions, and chemicals can be applied by the consumer to the appendage sleeve just before use.

In another embodiment, the additive is encapsulated and then applied to the product surface. Encapsulation is a process by which a material or mixture of materials is coated with or entrapped within another material or mixture of materials. The technique is commonly used in the food and pharmaceutical industries. The material that is coated or entrapped is normally a liquid, although it can also be a solid or gas, and is referred to herein as the core material. The material that forms the coating is referred to as the carrier material. A variety of encapsulation techniques are well-known in the art and can be used in the current invention, including spray drying, spray chilling and cooling, coacervation, fluidized bed coating, liposome entrapment, rotational suspension separation, and extrusion.

Spray-drying application techniques can be used. To prepare a material for spray drying, the carrier material is dissolved in an aqueous solution. The core ingredient is added to this solution and mixed thoroughly. A typical load of carrier to core material is 4:1, although much higher or lower loads can be used. The mixture is homogenized, and then fed into a spray dryer where it is atomized and released into a stream of hot air. The water is evaporated, leaving a dried particle comprising the core material trapped within the carrier matrix.

Suitable carrier materials include but are not limited to gums, gum Arabic, modified starches, gelatin, cellulose derivatives, and maltodextrins. Suitable core materials include but are not limited to flavors, natural oils, additives, sweeteners, stabilizers besides the other various additives mentioned above.

Regardless of the mechanism utilized to apply the chemical additives to the nonwoven substrate, the additives can be applied to the protective articles via an aqueous solution, non-aqueous solution, oil, lotion, cream, suspension, gel, etc. When utilized, an aqueous solution can contain any of a variety of liquids, such as various solvents and/or water. Moreover, the solution can often contain more than one additive. In some embodiments, the additives applied by an aqueous solution or otherwise constitute approximately less than 80% by weight of the appendage sleeve. In other embodiments, in order to maintain sufficient absorbency of the substrate, the additives can be applied in an amount less than about 50% of the weight of the substrate.

The present invention has been described in general and in detail by way of examples. The words used are words of description rather than of limitation. Persons of ordinary skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein and the appended claims should not be limited to the description of the preferred versions herein.

1. A protective article comprising: a nonwoven fiber web having cross-direction and machine direction stretchability; and a coating of a stable, non-leaching antimicrobial or antiviral composition over at least a portion of a first, exterior surface of said nonwoven fiber web, said antimicrobial or antiviral composition having a non-cytotoxic activity but a broad spectrum kill, and a therapeutic composition coating a second, interior surface adapted to contact mammalian skin.

2. The protective article according to claim 1, wherein said nonwoven fiber web is a laminate of a first nonwoven layer, an intermediate layer, and a second nonwoven layer.

3. The protective article according to claim 2, wherein said intermediate layer is either a unidirectional or bidirectional stretchable structural layer.

4. The protective article according to claim 2, wherein said intermediate later is a barrier film layer.

5. The protective article according to claim 1, wherein said antimicrobial composition includes polyhexamethylene biguanide (PHMB), and at least one or a combination of the following; chitosan, a chaotropic surfactant, polyols, copper oxide, and an organic acid.

6. The protective article according to claim 5, wherein said chaotropic surfactant includes an alkyl-polyglycoside.

7. The protective article according to claim 5, wherein said polyol includes xylitol.

8. The protective article according to claim 5, wherein said organic acid is either: benzoic acid, citric acid, acetic acid, ascorbic acid, glycolic acid, lauric acid, maleic acid, succinic acid, salicylic acid, or a combination thereof.

9. The protective article according to claim 5, wherein said copper oxide is either cupric or cuprous oxide, or both.

10. The protective article according to claim 1, wherein said therapeutic composition includes any one or combination of the following: anti-inflammatory agents, moisturizing agents, cationic polymers, vasodilators, corticosteroids, dimethyl sulfoxide (DMSO), capsaicin, menthol, methyl salicylate, DMSO/capsaicin, cationic polymers, anti-fungal agents, ZnO, or antioxidant agents.

11. The protective article according to claim 10, wherein said antioxidant agent either an organic or inorganic compound.

12. The protective article according to claim 11, wherein said organic compound is either super-oxide dismutase (SOD), glutathione, or a combination of both.

13. The protective article according to claim 11, wherein said inorganic compound is selenium or selenium derivatives.

14. The protective article according to claim 1, wherein said article includes an aromatherapy agent.

15. The protective article according to claim 1, wherein said article is one of the following: an external garment, glove, sock, foot cover, appendage sleeve, wrap or padding, face masks, or ear and head coverings.

16. A glove comprising: a hollow member defining an opening for receiving a hand therein, the hollow member having an interior surface configured to be placed adjacent to a hand when the glove is donned and an opposite exterior surface, the hollow member comprising an elastic laminate, the elastic laminate including at least one nonwvoven layer; and a stably-associated, non-leaching antimicrobial coating on at least a portion of said exterior surface and a therapeutic agent on said interior surface.

17. Be glove according to claim 16, wherein said antimicrobial coating includes polyhexamethylene biguanide (PHMB), and at least one or a combination of the following: chitosan, a chaotropic surfactant, polyols, copper oxide, and an organic acid.

18. The glove according to claim 16, wherein said therapeutic agent is a skin conditioner or an aromatherapy agent.

19. The glove according to claim 16, wherein said therapeutic agent includes any one or combination of the following: anti-inflammatory agents, moisturizing agents, cationic polymers, vasodilators, corticosteroids, dimethyl sulfoxide (DMSO), capsaicin, menthol, methyl salicylate, DMSO/capsaicin, cationic polymers, anti-fungal agents, ZnO, or antioxidant agents.

20. The glove according to claim 19, wherein said antioxidant agent either an organic or inorganic compound.

21. The glove according to claim 20, wherein said organic compound is either super-oxide dismutase (SOD), glutathione, or a combination of both.

22. The glove according to claim 20, wherein said inorganic compound is selenium or its derivatives.

23. The glove according to claim 16, wherein said exterior or interior surface includes a cleaning agent.

24. The glove according to claim 23, wherein said exterior or interior surface includes a skin cleaning agent.
